(19) United States
(12) Patent Application Publication
(10) Pub. No.: US 2006/0195178 A1
(43) Pub. Date: Aug. 31, 2006

(54) Aneurismal sack deflator

(76) Inventor: Stephen West, Pembroke Pines, FL (US)

Correspondence Address: George H. Gerstman;Seyfarth Shaw LLP, Suite 4200, 55 East Monroe Street, Chicago, IL, 60603, US

(21) Appl. No.: 11/069,003

(22) Filed: Feb. 28, 2005

(51) Int. Cl. A61F 2/06

(52) U.S. Cl. 623/1.39

(57) ABSTRACT

An aneurismal sack deflator is disclosed including a body having an aneurismal sack neck closure surface. An internal surface defines at least a portion of a fluid passageway, with the internal surface configured to provide a Venturi effect to thereby reduce fluid pressure in the fluid passageway. A semi-permeable region is in fluid communication with the fluid passageway and the aneurismal sack. The semi-permeable region permits first blood components to pass therethrough out of the aneurismal sack and into the fluid passageway when the lower surface is under reduced pressure and does not permit second blood components to pass therethrough.

### TECHNICAL FIELD

The present invention relates to devices which are implanted within a vascular system to impart structural integrity. In particular, the present invention relates to intravascular devices for implantation in a blood vessel having an aneurysm and deflation of an aneurismal sack.

### BACKGROUND

An aneurysm is an occurrence in which an abnormal enlargement or dilation of a portion of a blood vessel caused by damage to or weakness in the blood vessel wall. Although aneurysms can occur in any type of blood vessel, they most frequently form in an artery. Aneurysms pose a significant risk because the blood pressure within the blood vessel could result in a rupture of the blood vessel wall, causing potentially life-threatening bleeding.

One traditional approach to treating aneurysms is to utilize intravascular prosthetic devices, such as stents, and placing such devices in the blood vessel lumina. The procedure is generally performed to seal off a vascular leak, false aneurysm or arteriovenous communication or to create an internal bypass in atherosclerotic aneurysms. Intravascular stents having a constricted diameter for delivery through a blood vessel and an expanded diameter for applying a radially outwardly extending force for treating the aneurysm in a blood vessel are known in the art. These stents are usually covered with a low friction material and operate as a substitute for the aneurismal wall of the blood vessel, alleviating pressure on the aneurismal wall by isolating the aneurysm from blood flow within the vessel. A deficiency of these prior art devices is that poor positioning of the prosthetic in relation to the walls of the affected blood vessel can permit blood flow between the prosthetic and the aneurysm, thereby creating pressure on the aneurismal wall that may be sufficient to burst the blood vessel wall of the aneurysm.

A technique for handling this difficulty is to incorporate a stent-like insert in each of the two ends of the prosthetic in order to force or bias the prosthetic against the blood vessel wall so as to attempt to form a closer-fitting seal between the prosthetic and the treated blood vessel wall. The difficulty with this method is that larger blood components may leak into the aneurismal sack.

Therefore, it would be advantageous to have a prosthetic for treating a blood vessel having an aneurysm that reduces pressure on the aneurismal sack wall, siphons the blood out of the aneurismal sack, and minimizes the risk that larger blood components will leak back into the aneurismal sack and impart pressure.

The disclosed aneurismal sack deflator is a significant enhancement of the typical construction of conventional prosthetics, wherein a semi-permeable membrane positioned in fluid communication with the aneurismal sack permits fluid to pass out of the aneurismal sack and does not permit larger blood components to enter the aneurismal sack.

### SUMMARY

The illustrative embodiment of the present invention relates to an aneurismal sack deflator including a body having an aneurismal sack neck closure surface. An internal surface defines at least a portion of a fluid passageway, with the internal surface configured to provide a Venturi effect to thereby reduce fluid pressure in the fluid passageway. A semi-permeable region is in fluid communication with the fluid passageway and the aneurismal sack. The semi-permeable region permits first blood components to pass therethrough out of the aneurismal sack and into the fluid passageway when the lower surface is under reduced pressure and does not permit second blood components to pass therethrough.

The invention provides an aneurismal sack deflator for imparting structural integrity to a blood vessel having an aneurysm, that siphons the blood out of the aneurismal sack, reducing pressure therein, and eliminates the risk that blood will leak back into the aneurismal sack and impart pressure.

A more detailed explanation of the invention is provided in the following description and claims and is illustrated in the accompanying drawings.

### BRIEF DESCRIPTION OF THE DRAWINGS

While the drawings depict preferred embodiments of the present invention, they are by way of example only and are not intended to limit the scope of the invention. It is expected that variations and further modifications as well as further applications of the principles will occur to others skilled in the art and while differing from the foregoing, remain within the spirit and scope of the invention as described.

FIG. 1 is an isometric view of an embodiment of an aneurismal sack deflator;

FIG. 2 is a side view of the aneurismal sack deflator in accordance with FIG. 1;

FIG. 3 is a side view of the aneurismal sack deflator after the aneurismal sack has been collapsed; and

FIG. 4 is a side view of an alternative embodiment of an aneurismal sack deflator of FIG. 1, wherein the aneurismal sack deflator is biodegradable.

### DETAILED DESCRIPTION

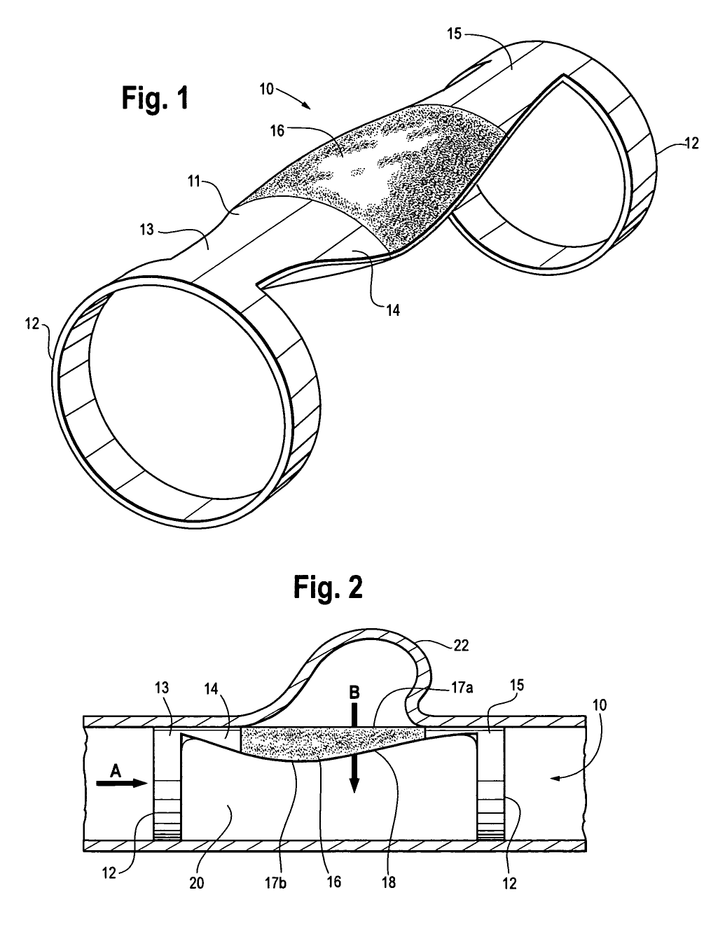

Referring now to FIG. 1, the preferred embodiment of the present invention is illustrated as aneurismal sack deflator 10. Aneurismal sack deflator 10 includes a body 11 having struts 12, first end 13, aneurismal sack neck closure surface 14 and second end 15. It is preferred that aneurismal sack neck closure surface 14 incorporate a semi-permeable region 16 positioned between first end 13 and second end 15.

Semi-permeable region 16 is preferably permeable to certain relatively smaller blood components, including, but not limited to, blood plasma, blood clotting factors, sugars, lipids, vitamins, minerals, hormones, enzymes, antibodies, and other proteins but not to permeable to other usually larger blood components, such as red blood cells, white blood cells, and platelets.

Additionally, semi-permeable region 16 is preferably a membrane or graft that has appropriate selective filtering properties, is composed of any suitable material that is biocompatible, has appropriate stiffness and flexibility, and is permeable to smaller blood components but impermeable to larger blood components, including, but not limited to, regenerated cellulose, cellulose acetate, cellulose diacetate, polysulfone, polycarbonate, polyethylene, porous polyethylene, polyolefin, polypropylene, polyvinylidene fluoride, polyvinylchloride, polymethylmethacrylate and polyethylenevinylalocohol. For example, semi-permeable region 16 may be a medical grade mesh material with a pore size of 6-8 μm, so as to prevent the passage of red blood cells therethrough.

As illustrated in FIG. 2, in accordance with a preferred embodiment, aneurismal sack neck closure surface 14 has semi-permeable region 16. It is positioned adjacent to an aneurismal sack 22 and defines at least a part of a fluid passageway 20 and is supported therein by struts 12. Aneurismal sack deflator 10 includes an internal surface 18 opposite of aneurismal sack neck closure surface 14. Surface 18 is shaped to provide a Venturi effect (discussed in detail below) and thereby increase the velocity of blood flowing in direction “A” through fluid passageway 20. In one embodiment internal surface 18 is substantially curvilinear in shape, with the greatest distance between internal surface 18 and aneurismal sack neck closure surface 14 at approximately the midpoint between first end 13 and second end 15. The form of internal surface 18 is critical to the operation of aneurysm deflator 10. As the velocity of the fluid (blood) increases in fluid passageway 20, the static pressure of the fluid drops. Such a reduction of static pressure with increasing velocity is known as the “Venturi effect”, and results in a pressure within fluid passageway 20 that is decreased as compared to the pressure outside of fluid passageway 20.

The incorporation of curved internal surface 18 creates a constricted, throat-like portion that increases the velocity and lowers the pressure of the fluid in fluid passageway 20 to provide the Venturi effect. The structure is similar in cross-section to a wing or a hydrofoil, which has a higher degree of curvature on a first surface than on a second surface. When a fluid moves over the surfaces of such an object, the flow rate is higher over the surface of greatest curvature. The resulting differential in pressures between the more curved surface and less curved surface yields suction pressure, or pressure against the less curved surface.

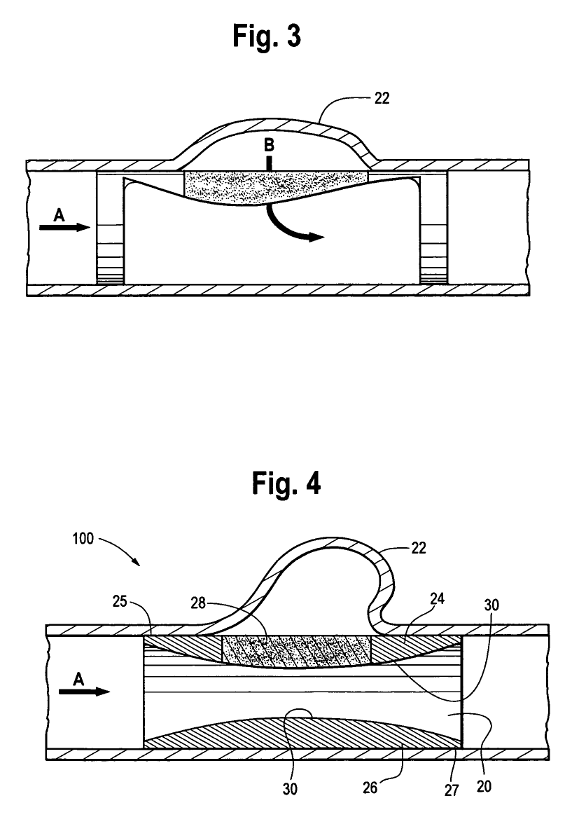

Likewise, as the blood moves through fluid passageway 20 and over internal surface 18, a pressure differential results between the fluid contained in aneurismal sack 22 and the fluid flowing through fluid passageway 22. This pressure differential causes the fluid in aneurismal sack 22 to flow out of aneurismal sack 22 and into fluid passageway 20 through semi-permeable membrane 16 along fluid path “B” in order to equalize the pressures of aneurismal sack 22 and fluid passageway 20. Semi-permeable region 16 is in fluid communication with aneurismal sack 22 along upper surface 17a and fluid passageway along lower surface 17b in order to allow blood components (not shown) to flow therethrough out of aneurismal sack 22 and into fluid passageway 20 along fluid path “B”, resulting in a deflation of aneurismal sack 22 due the decrease in fluid pressure therein as illustrated in FIG. 3.

Additionally, body 11 may incorporate holes or ports (not shown) therethrough, perpendicular to fluid passageway 20 to further facilitate fluid flow out of aneurismal sack 22 and into fluid passageway 20. Body 11 may substitute semi-permeable region 16 with a mechanical-type check valve (not shown), moveable between an open position and a closed position. Such a check valve would be in the open position so long as fluid remained in aneurismal sack 22 in an amount sufficient to place enough pressure on the check valve to maintain an open position.

Referring now to FIG. 4, an alternative embodiment is illustrated as biodegradable aneurismal sack deflator 100. Biodegradable aneurismal sack deflator 100 includes biodegradable aneurismal sack neck closure member 24 and biodegradable lower member 26. Biodegradable aneurismal sack neck closure member 24 and biodegradable lower member 26 may be semi-permeable. The biodegradable material forming biodegradable aneurismal sack deflator 100 should have a high biocompatibility with minimal inflammatory response, as well as a suitable biodegradation period, for example poly-l-lactic acid. Alternatively, biodegradable aneurismal sack neck closure member 28 can incorporate a semi-permeable region 28. The semi-permeable region 28 has appropriate selective filtering properties, composed of any suitable material that is biocompatible and biodegradable. The semi-permeable region 28 has appropriate stiffness and flexibility, and is permeable to smaller blood components but impermeable to larger blood components.

Biodegradable aneurismal sack deflator 100 with aneurismal sack neck closure member 24 having semi-permeable region 28 therein is positioned adjacent to aneurismal sack 22 in fluid passageway 20. Both biodegradable aneurismal sack neck closure member 24 and biodegradable lower member 26 include internal surfaces 30, 32 that are shaped to provide a Venturi effect by increasing the velocity of blood flowing in direction “A” through fluid passageway 20. In an alternative embodiment, internal surfaces 30, 32 are substantially curvilinear in shape, with the shortest distance between internal surface 30 of biodegradable aneurismal sack neck closure member 24 and internal surface 32 of biodegradable lower member 26 at approximately the midpoint between first end 25 and second end 27. The form of internal surfaces 30, 32 is critical to the operation of biodegradable aneurismal sack deflator 100. As stated above with reference to FIGS. 2 and 3, as the velocity of the fluid (blood) increases in fluid passageway 20, the static pressure of the fluid drops, thereby providing the Venturi effect. The incorporation of curved internal surfaces 30, 32 creates a more constricted, throat-like portion in fluid passageway 20 that further enhances the Venturi effect by increasing the velocity and lowering the pressure of the fluid in fluid passageway 20, resulting in deflation of aneurismal sack 22 due the decrease in fluid pressure therein.

While the invention has been described in conjunction with a preferred embodiment, it will be apparent to one skilled in the art that other objects and refinements of the disclosed aneurysm deflator may be made within the purview and scope of the subject matter to be protected.

The aneurismal sack deflator, in its various aspects and disclosed forms, is well adapted to the attainment of the stated features and advantages of others. The disclosed details are not to be taken as limitations of the subject matter sought to be protected, except as those details may be included in the appended claims. The embodiments in which an exclusive property or privilege is claimed are as follows:

What is claimed is:

1. An aneurismal sack deflator comprising:
a body having an aneurismal sack neck closure surface and an internal surface defining at least a portion of a fluid passageway, the internal surface configured to provide a Venturi effect to thereby reduce fluid pressure in the fluid passageway; and
said body having a semi-permeable region in fluid communication with the fluid passageway and the aneurismal sack, the semi-permeable membrane permitting first blood components to pass therethrough out of the aneurismal sack and into the fluid passageway when the lower surface is under reduced pressure and not permitting second blood components to pass therethrough.

2. The aneurismal sack deflator according to claim 1, wherein the body further includes at least one strut.

3. The aneurismal sack deflator according to claim 1, wherein the internal surface is substantially curvilinear.

4. The aneurismal sack deflator according to claim 1, wherein the semi-permeable region is selected from the group consisting of regenerated cellulose, cellulose acetate, cellulose diacetate, polysulfone, polycarbonate, polyethylene, porous polyethylene, polyolefin, polypropylene, polyvinylidene fluoride, polyvinylchloride, polymethylmethacrylate and polyethylenevinylalocohol.

5. The aneurismal sack deflator according to claim 1, wherein the semi-permeable region has a pore size of less than about 6-8 μm.

6. The aneurismal sack deflator according to claim 1, wherein the first blood components are selected from the group consisting of blood plasma, blood clotting factors, sugars, lipids, vitamins, minerals, hormones, enzymes, antibodies, and proteins.

7. The aneurismal sack deflator according to claim 1, wherein the second blood components are selected from the group consisting of red blood cells, white blood cells, and platelets.

8. The aneurismal sack deflator according to claim 1, wherein the passage of the first blood components out of the aneurismal sack through the semi-permeable membrane deflates the aneurismal sack.

9. A biodegradable aneurismal sack deflator comprising:
a biodegradable aneurismal sack neck closure member, a first end, a second end, and a first internal surface defining at least a portion of a fluid passageway, the internal surface configured to reduce fluid pressure in the fluid passageway; and
a biodegradable lower member having a second internal surface defining at least a portion of the fluid passageway, the second internal surface configured to provide a Venturi effect to thereby reduce fluid pressure in the fluid passageway.

10. The biodegradable aneurismal sack deflator according to claim 9, wherein the biodegradable aneurismal sack neck closure member is semi-permeable.

11. The biodegradable aneurismal sack deflator according to claim 9, wherein the first internal surface is substantially curvilinear.

12. The biodegradable aneurismal sack deflator according to claim 10, wherein the biodegradable aneurismal sack neck closure member permits first blood components to pass therethrough out of the aneurismal sack and into the fluid passageway when the first internal surface is under reduced pressure and does not permit second blood components to pass therethrough.

13. The biodegradable aneurismal sack deflator according to claim 10, wherein the biodegradable lower member is semi-permeable.

14. The biodegradable aneurismal sack deflator according to claim 9, wherein the second internal surface is substantially curvilinear.

15. The biodegradable aneurismal sack deflator according to claim 9, wherein the biodegradable aneurismal sack neck closure member includes a semi-permeable region having an upper surface and a lower surface positioned between the first end and second end in fluid communication with the fluid passageway and the aneurismal sack.

16. The biodegradable aneurismal sack deflator according to claim 15, wherein the semi-permeable region permits first blood components to pass therethrough out of the aneurismal sack and into the fluid passageway when the lower surface is under reduced pressure and does not permit second blood components to pass therethrough.

17. A method of deflating an aneurismal sack, comprising;
inserting aneurismal sack deflator in a blood vessel having an aneurismal sack, the aneurismal sack deflator having an internal surface configured to provide a Venturi effect to thereby reduce fluid pressure in the fluid passageway and a semi-permeable region;
orienting the aneurismal sack deflator to place the semi-permeable region in fluid communication with the aneurismal sack and the fluid passageway; and
passing first blood components out of the aneurismal sack and into the fluid passageway through the semi-permeable region when the internal surface is under reduced pressure from the Venturi effect; and
preventing second blood components from passing through the semi-permeable region.
